(19) United States
(12) Patent Application Publication
(10) Pub. No.: US 2012/0158139 A1
(43) Pub. Date: Jun. 21, 2012

(54) Method and Apparatus for a Porous Orthopedic Implant

(75) Inventor: James Jenq Liu, Mason, OH (US)

(73) Assignee: BIO2 TECHNOLOGIES, INC., Woburn, MA (US)

(21) Appl. No.: 13/326,665

(22) Filed: Dec. 15, 2011

Related U.S. Application Data
(60) Provisional application No. 61/424,321, filed on Dec. 17, 2010.

(51) Int. Cl. A61F 2/28; B05D 5/00; B05D 3/02

(52) U.S. Cl. 623/16.11; 427/2.26

(57) ABSTRACT

An orthopedic implant having a pyrolytic carbon composition is provided with a porous coating. The porous coating is bonded to the pyrolytic carbon implant using a bond coat that is reaction-bonded to the carbon material. The porous coating can be reaction-bonded to the bond coat to provide a porous structure having a structure that is conducive to the ingrowth of living tissue when implanted in the body.

### CROSS-REFERENCE TO RELATED APPLICATIONS

This application claims the benefit of Provisional Application No. 61/424,321 filed Dec. 17, 2010, the entire contents of which are incorporated herein by reference.

### FIELD OF THE INVENTION

The present invention relates generally to orthopedic implants and more specifically, to an orthopedic implant having a porous layer on an interfacial coating.

### BACKGROUND OF THE INVENTION

Orthopedic implants, such as femoral stems, acetabular cups, knee replacements, and the like typically require a biocompatible porous layer to promote ingrowth of bone tissue from living tissue surrounding the implant site. Ingrowth of healthy living tissue is essential to ensure fixation of the implant for long-term, if not permanent, use. Poor fixation results in loosening of the implant which then requires revision surgery to repair or replace the implant at high cost and extreme discomfort to the patient.

Various methods are known in the art for providing a porous coating on an orthopedic implant but there has yet to be provided a porous coating and a method of providing a porous coating that effectively adheres a porous coating of fiber or wire-based materials at low cost.

### BRIEF SUMMARY OF THE INVENTION

The present invention meets the objectives of an effective orthopedic implant that provides a method of forming an orthopedic implant with a porous coating that provides healthy tissue ingrowth.

According to an embodiment of the invention, a method of forming an orthopedic implant in a pyrolytic carbon composition with a porous coating is provided. The method includes applying a bond coat and sintering the coated implant for the bond coat to react with the non-porous surface of the pyrolytic carbon implant to provide a coated surface. A porous material comprising fiber is applied to the coated surface and sintered to react the porous material at a reaction temperature so that the porous material comprising fiber reacts with the bond coat on the coated surface to provide a porous coating adhered on the implant.

According to another embodiment of the invention, an orthopedic implant is provided that has a biocompatible orthopedic core implant of pyrolytic carbon with a bond coat adhered to at least one surface of the implant and a porous coating comprising intertangled and bonded fiber segments adhered to the bond coat.

According to another embodiment of the invention, a method of forming an orthopedic implant is provided. In this embodiment, a silicon coating is applied to a pyrolytic carbon implant, with a fiber-based coating applied to the silicon coating. The coated implant is sintered to react the silicon coating with the pyrolytic carbon implant to provide a silicon coating bonded to a coated surface of the implant and to reaction-bond the fiber-based coating to the silicon coating, to provide a porous coating on the orthopedic implant. Alternative embodiments of the invention include pore former components in the fiber-based coating that define the pore size and pore size distribution of the porous coating.

### BRIEF DESCRIPTION OF THE SEVERAL VIEWS OF THE DRAWING

The foregoing and other objects, features, and advantages of the invention will be apparent from the following detailed description of the several embodiments of the invention as illustrated in the accompanying drawings in which like reference characters refer to the same parts throughout the different views. The drawings are not necessarily to scale, with emphasis instead being placed upon illustrating the principles of the invention.

FIG. 1 shows a carpometacarpal implant with a porous coating according to the present invention.

FIG. 2 is an exploded view of the implant of FIG. 1 depicting the porous coating of the present invention.

FIG. 3 is a flow chart depicting a method according to the present invention.

FIG. 4 depicts an XRD analysis of the surface of an implant according to the present invention.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION OF THE INVENTION

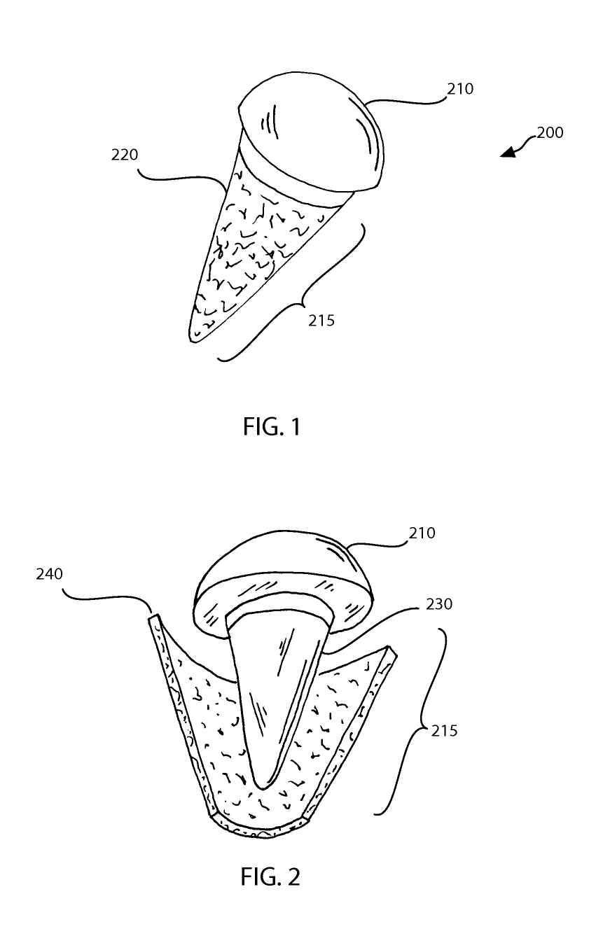

The present invention provides a method and apparatus for a porous orthopedic implant. FIG. 1 depicts an exemplary carpometacarpal implant that is used in resection arthroplasty for thumb trapeziometacarpal arthritis in a pyrolytic carbon (pyrocarbon) composition. Those skilled in the art will appreciate that the present invention may be applied to numerous other prosthetic implants that can be improved through the use of a porous region or surface into which tissue can in-grow post operatively, such as intervertebral devices for spinal stabilization, femoral condylar knee implants, and femoral hip stem implants to name only a few. The carpometacarpal implant 200 shown in FIG. 1 includes a spherical head 210 and distal taper 215 section upon which a porous surface 220 is shown that is inserted into the intramedullary canal when implanted during the resection arthroplasty. Ideally, a thumb carpometacarpal implant must be strong and stable, provide full range of motion, and prevent loosening, that in combination have not been previously provided by known implants. Pyrolytic carbon carpometacarpal implants typically provide a high strength and biocompatible material with a hard, wear resistant surface that readily provides full range of motion. Pyrolytic carbon implants typically do not have porous surfaces into which tissue can grow and integrate with surrounding tissue at the implant site. The present invention, however, provides a porous surface that can be bonded to pyrolytic carbon to provide a porous surface that exhibits a high degree of osteointegration and tissue ingrowth using biocompatible and/or bioresorbable materials.

Pyrolytic carbon is a type of turbostratic carbon that has a similar structure as graphite, consisting of carbon atoms covalently bonded in hexagonal arrays. The arrays are stacked and held together by weak interlayer binding, but with disordered layers that give pyrolytic carbon increased durability compared to graphite. The material is biocompatible in that it does not elicit adverse reactions when implanted into human bodies, and the material is well suited for small orthopedic joints such as fingers and spinal inserts.

FIG. 2 depicts an exploded view of the carpometacarpal implant 200 of FIG. 1 that shows a spherical head 210 and a bond coat 230 applied to a distal taper 215 surface upon which the porous surface 220 is desired. A porous material 240 is applied to the bond coat 230 surface to provide the porous surface 220. The bond coat 230 is adhered to the body 210 and the porous material is bonded to the bond coat 230 as is further described hereinafter.

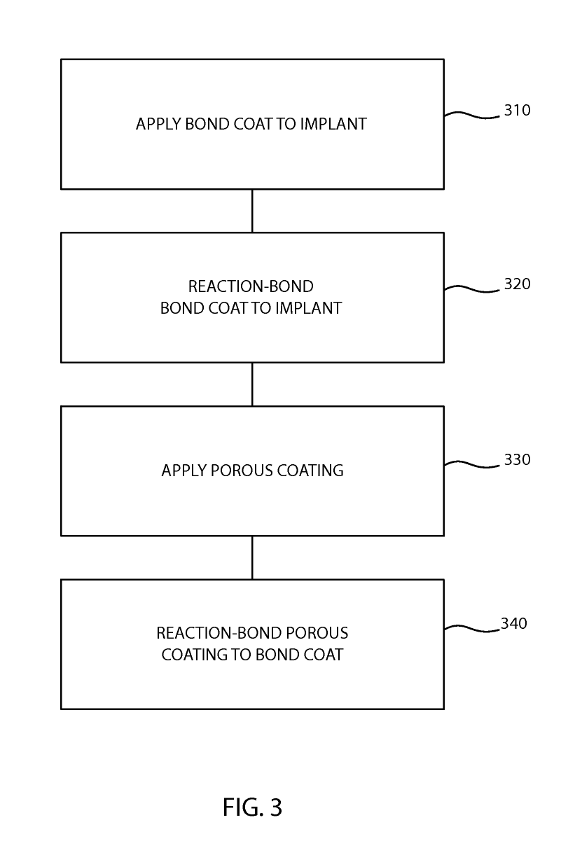

FIG. 3 depicts a flowchart of the method of fabricating the porous orthopedic implant according to the present invention. A biocompatible synthetic prosthesis upon which a porous coating is applied according to the present invention is provided. The biocompatible synthetic prosthesis can be any one of a number of synthetic prostheses such as the carpometacarpal implant 200 according to FIG. 1. Biocompatible synthetic prosthetic devices of the present invention are composed of pyrolytic carbon or pyrocarbon materials. Other biocompatible materials can be used such as titanium, tantalum, silicon nitride, stainless steel, cobalt chromium, polymeric materials, ceramics, or other compositions that are biologically neutral and generally biologically inert with respect to living tissue.

At step 310 a bond coat is applied to the implant at the surface or region of the implant upon which a porous coating is desired. The selection of the composition and characteristics of the bond coat is dependent upon the composition and characteristics of the implant and the composition and characteristics of the porous coating, as further described herein.

In the exemplary embodiment, the implant is a carpometacarpal implant as shown in FIG. 1 with the spherical head 210 and distal taper 215 having a pyrolytic carbon composition and the porous material 240 including silicon carbide fiber. The bond coat composition in this exemplary embodiment can be a colloidal suspension of silicon nanopowder in methanol or water solvents forming a suspension that can be applied to the implant. At step 310 the bond coat is applied by immersion, brush, spray, or other application method of a liquid solution. The bond coat is dried at room temperature or the bond coat drying can be accelerated at elevated temperatures.

At step 320 the bond coat is reaction-bonded to the implant. In the exemplary embodiment the coated implant is heated to 1,400° C. for two hours in an inert environment attained by purging argon in a kiln or furnace, though other inert environment chambers such as inert gas purged or a vacuum environment are suitable. Alternatively, the bond coat can include small amounts such as approximately 3-5% organic binder to modify the viscosity of the coating for application and to promote adhesion until the bond coat is reaction-bonded to the implant at step 320. When additives such as an organic binder are included the heating step can be adjusted to dwell at approximately 350° C. for a period of time to sufficiently decompose and remove the binder additives before heating to the appropriate reaction-bond temperature.

In the exemplary embodiment, the silicon in the bond coat reacts with the carbon in the pyrolytic carbon implant to form silicon carbide at the interfacial layer between the bond coat and the implant thereby forming a strong bond between the bond coat and the implant. Reaction-formation of silicon and carbon into silicon carbide in this embodiment occurs at a reaction formation temperature of at least 1,400° C. Implants of compositions other than pyrolytic carbon can be used as an alternative embodiment, such as ceramic materials such as alumina and zirconia. In these alternate embodiments, a bond coat of silicon applied to a ceramic material will react with the ceramic material to form glass or glass-ceramic that adheres the silicon bond coat to the surface of the implant.

At step 330 the porous coating is applied to the implant where the bond coat 230 is applied. In the exemplary embodiment the porous coating applied to the implant is carbon fiber mixed in a plastically formable batch composition consisting of chopped carbon fiber, an organic binder, and a liquid. The plastically formable batch composition can be directly applied to the implant at step 330 spread to a thickness of approximately 1-2 millimeters. Alternatively, the batch material can be formed or extruded into a ribbon or sheet of approximately 1-2 millimeters thickness to coat the portion of the implant where the bond coat 230 is applied.

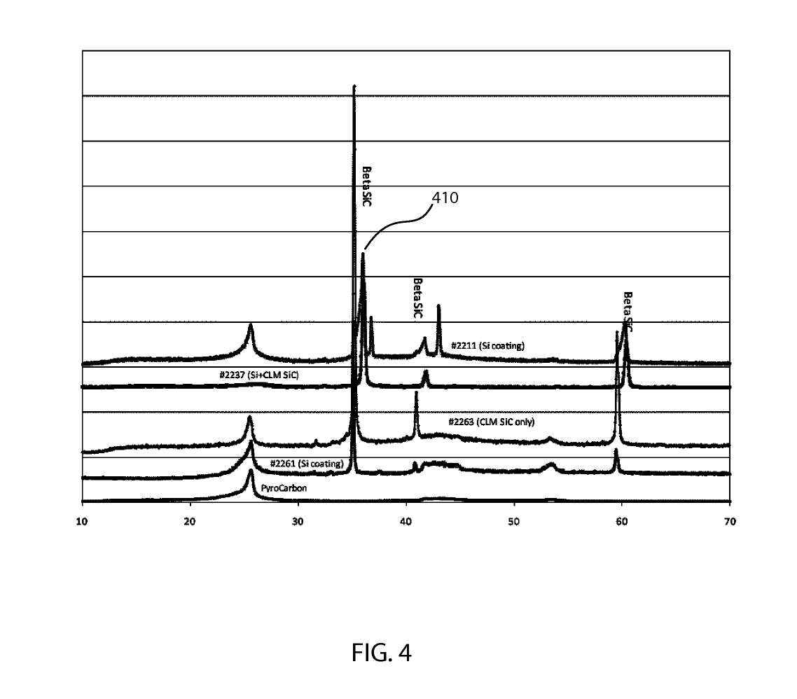

At step 340 the porous coating is reaction-bonded to the bond coat. The coated implant is heated to a temperature of at least 350° C. for approximately one hour to thermally decompose the organic binder material leaving the carbon fiber in direct contact with the silicon material of the bond coat 230. The coated implant is then heated to approximately 1,400° C. in an inert environment, such as a vacuum kiln or an argon or similar inert gas-purged environment that would permit the reaction of carbon with silicon to reaction-form silicon carbide composition in the fibers and the interfacial layer between the fibers and the implant. An XRD analysis of the surface of an illustrative example of the exemplary embodiment is shown at FIG. 4, where a silicon carbide peak is clearly shown within the porous layer on the surface of the pyrolytic carbon implant. In an alternate embodiment, the batch composition can include additional quantities of silicon powder that would be available to fully react with the carbon fiber during the high temperature curing process wherein the bond coat reacts with the fibers to form a porous layer. In yet another embodiment, excess silicon material can be included in the batch composition so that more silicon than necessary is available to fully react with the carbon fibers and the bond coat to form a silicon bonded silicon carbide porous layer. Subsequent heating in an oxygen environment will oxidize the excess silicon to form a silica compound in the porous layer to enhance the biocompatibility of the coated implant. The resulting structure is a porous coating of intertangled fibers having a composition of silicon carbide with pore space defined by the spacing between the fibers, with at least a portion of the fibers bonded at the bond coat 230 interface. The porous coating is a substantially rigid matrix of intertangled fibers that are bonded together at intersecting and overlapping regions between adjacent fibers.

Volatile pore former components can be included in the plastically formable batch composition that can provide for increased porosity by predetermining minimum spacing between adjacent fibers. The volatile pore former components in the plastically formable batch composition are mixed and distributed throughout the batch composition and fiber. When the porous coating is reaction-bonded to the bond coat at step 340, the volatile pore former component is thermally decomposed during the heating step via pyrolysis or by thermal degradation or volatilization, leaving a void in the mixture that becomes pore space in the resulting structure. Pore former components can include microwax emulsions or phenolic resin particles of a specific size and size distribution, or other organic particles of a specific size and shape to provide porosity having a pore size distribution that can promote osteoconduction and ingrowth of living tissue.

In a second exemplary embodiment a carpometacarpal implant composed of pyrolytic carbon as shown in FIG. 1 has a bond coat 230 of silicon with a porous material 240 of hydroxyapatite with fiber. In this embodiment, the silicon bond coat is applied as described above with respect to the first exemplary embodiment. The silicon bond coat is applied and an interfacial layer of silicon carbide is formed to bond the silicon bond coat to the pyrolytic carbon implant. The residual silicon of the bond coat remains on the exposed coated surface, though it is acceptable for at least a portion of the outer layer of the bond coat to oxidize into silica due to exposure to ambient air. Hydroxyapatite is applied to the surface of the distal taper 215 and sintered at 1,250° C. for approximately four hours to bond the hydroxyapatite to the silicon layer of the bond coat.

Alternative embodiments are contemplated that include hydroxyapatite as applied in the second exemplary embodiment with a layer of fiber having a diameter of 2 μm to about 60 μm with a length of about 0.045 inches applied to the hydroxyapatite-coated implant. In these alternative embodiments, the fiber composition can be silicon carbide, silicon nitride, ceramic, glass or hydroxyapatite fiber. The coated implant is heated to about 1,250° C. to create a porous layer comprising fiber bonded with hydroxyapatite. In this alternate embodiment, volatile pore former components can be included with the fiber material that can provide for increased porosity in the porous coating by predetermining minimum spacing between adjacent fibers. The volatile pore former components are thermally decomposed during the heating step via pyrolysis or by thermal degradation or volatilization, leaving a void in the structure that can promote the ingrowth of living tissue when implanted in bone. These pore former components can include phenolic resins, carbon particles, or polymethyl methacrylate particles just to name a few. A pore former component can be any material that is non-reactive with the composition of the fiber, the bond coat and/or the composition of the implant upon which a porous coating is applied.

Alternative embodiments are contemplated that include the hydroxyapatite and fiber in a composition of silicon carbide, silicon nitride, ceramic, glass, or hydroxyapatite, forming a mixture applied directly to the silicon bond coat layer applied to the implant. In this embodiment, the silicon bond coat is applied as described above with respect to the first exemplary embodiment. A plastic mixture is formed of hydroxyapatite and the fiber material, the fiber having an average diameter of approximately 2 μm to about 60 μm with a length of about 0.045 inches, the mixture having a ratio of hydroxyapatite to fiber in the range of about 2:1 by weight, with a small amount of HPMC as an organic binder and water. The plastic mixture is applied to the silicon bond coat layer applied to the implant, and cured. The curing step heats the coated implant to about 1,250° C. to bond the fiber within the hydroxyapatite matrix that is bonded to the silicon bond coat layer to provide a porous coating on the implant.

In a third exemplary embodiment, a carpometacarpal implant composed of pyrolytic carbon as shown in FIG. 1 is formed with a porous coating of silicon carbide, silicon nitride, ceramic, glass or hydroxyapatite fiber in a matrix of hydroxyapatite. In this embodiment, a bond coat 230 of silicon is applied to the distal taper 215 by immersion, brush, spray, or other application method of a liquid solution. The bond coat is dried at room temperature or the bond coat drying can be accelerated at elevated temperature. A plastic mixture of hydroxyapatite with silicon carbide, silicon nitride, ceramic, glass, or hydroxyapatite fiber is prepared, the fiber having an average diameter of approximately 2 μm to about 60 μm with a length of about 0.045 inches, the mixture having a ratio of hydroxyapatite to fiber in the range of about 2:1 by weight, with a small amount of HPMC as an organic binder and water. The plastic mixture is applied to the distal taper 215 of the implant on the dried silicon bond coat layer. The coated implant is cured at 1,400° C. for two hours in an inert environment attained by purging argon in a kiln or furnace, though other inert environment chambers such as inert gas purged or a vacuum environment are suitable. In this way, the silicon layer of the bond coat forms an interfacial layer of silicon carbide, bonding the bond coat to the distal taper 215 of the implant at the same time the fibers and hydroxyapatite matrix are bonded to the bond coat layer, resulting in a porous coating on the distal taper 215 of the implant.

In this embodiment, volatile pore former components can be included plastic mixture that can provide for increased porosity in the porous coating by predetermining minimum spacing between adjacent fibers. The volatile pore former components are thermally decomposed during the heating step via pyrolysis or by thermal degradation or volatilization, leaving a void in the structure that can promote the ingrowth of living tissue when implanted in bone. These pore former components can include phenolic resins, carbon particles, or polymethyl methacrylate particles just to name a few. A pore former component can be any material that is non-reactive with the composition of the fiber, the bond coat and/or the composition of the implant upon which a porous coating is applied.

The present invention has been herein described in detail with respect to certain illustrative and specific embodiments thereof, and it should not be considered limited to such, as numerous modifications are possible without departing from the spirit and scope of the appended claims.

What is claimed is:

1. A method of forming an orthopedic implant with a porous surface, the method comprising:
providing a pyrolytic carbon orthopedic implant having a non-porous surface;
applying a bond coat on the non-porous surface;
adhering the bond coat on the non-porous surface of the pyrolytic carbon implant to provide a coated surface;
applying a porous material comprising fiber to the coated surface; and
adhering the porous material on the coated surface to provide a porous coating.

2. The method according to claim 1 wherein the porous material further comprises a pore former.

3. The method according to claim 2 wherein the step of adhering the porous material on the coated surface includes sintering the porous material to remove the pore former and adhere the porous material on the coated surface.

4. The method according to claim 3 wherein sintering the porous material to remove the pore former comprises thermally decomposing the pore former.

5. The method according to claim 1 wherein the bond coat is a liquid and the step of applying a bond coat comprises at least one of immersion, spray, and brush application.

6. The method according to claim 1 wherein the bond coat comprises a colloidal suspension of silicon.

7. The method according to claim 6 wherein the bond coat further comprises an organic binder in an amount ranging from about 3% to about 5% by weight.

8. The method according to claim 1 wherein the orthopedic implant is a carpometacarpal implant.

9. An orthopedic implant comprising:
a biocompatible orthopedic core implant of pyrolytic carbon having at least one surface;
a bond coat adhered to the at least one surface of the orthopedic implant; and
a porous coating comprising intertangled and bonded fiber segments, the porous coating adhered to the bond coat.

10. The orthopedic implant according to claim 9 wherein the bond coat comprises silicon.

11. The orthopedic implant according to claim 9 wherein the bonded fiber segments have a composition comprising silicon carbide.

12. The orthopedic implant according to claim 9 wherein the bonded fiber segments have a composition comprising at least one of silicon carbide, silicon nitride, ceramic, glass, and hydroxyapatite.

13. The orthopedic implant according to claim 9 further comprising a coating of hydroxyapatite.

14. The orthopedic implant according to claim 13 wherein the bonded fiber segments have a composition comprising one of silicon carbide, silicon nitride, ceramic, glass and hydroxyapatite.

15. The orthopedic implant according to claim 9 wherein the orthopedic implant is a carpometacarpal implant.

16. A method of forming an orthopedic implant comprising:
providing a pyrolytic carbon implant;
applying a silicon coating on the pyrolytic carbon implant;
applying a fiber-based coating to the silicon coating; and
heating the pyrolytic carbon implant to react the silicon coating with the pyrolytic carbon implant to provide a silicon coating bonded to a surface of the pyrolytic carbon implant and reaction-bond the fiber-based coating to the silicon coating.

17. The method according to claim 16 wherein the fiber-based coating further comprises a pore former.

18. The method according to claim 17 wherein the step of heating the pyrolytic carbon implant includes heating the fiber-based coating to remove the pore former.

19. The method according to claim 18 wherein heating the fiber-based coating to remove the pore former comprises thermally decomposing the pore former.

20. The method according to claim 16 wherein the silicon coating is a liquid and the step of applying a silicon coating comprises at least one of immersion, spray, and brush application.

21. The method according to claim 20 wherein the silicon coating comprises a colloidal suspension of silicon.

22. The method according to claim 20 wherein the silicon coating further comprises an organic binder in an amount ranging from about 3% to about 5% by weight.

23. The method according to claim 16 wherein the orthopedic implant is a carpometacarpal implant.
